(19) United States
(12) Patent Application Publication
(10) Pub. No.: US 2013/0005714 A1
(43) Pub. Date: Jan. 3, 2013

(54) Water-Soluble Meloxicam Granules

(75) Inventors: Stefan HENKE, Gau-Odernheim (DE); Martin Andreas FOLGER, Ingelheim (DE); Jens SCHMALZ, Hueffelsheim (DE); Diana KEILHOFER, Mainz (DE); Hans-Juergen KROFF, Schoeneberg (DE); Nina HERZ, Windesheim (DE)

(73) Assignee: BOEHRINGER INGELHEIM VETMEDICA GMBH, Ingelheim (DE)

(21) Appl. No.: 13/604,266

(22) Filed: Sep. 5, 2012

Related U.S. Application Data
(63) Continuation of application No. 10/694,569, filed on Oct. 27, 2003.

(30) Foreign Application Priority Data
Oct. 25, 2002 (DE) 102 50 081
Oct. 25, 2002 (DE) 102 50 081.9

(51) Int. Cl. A61K 31/5415; A61P 29/00; B05D 1/02; A61P 11/00

(52) U.S. Cl. 514/226.5; 427/2.14

(57) ABSTRACT

Water soluble meloxicam granules comprising:
(a) meloxicam;
(b) a salt forming agent which forms the meglumine, sodium, potassium, or ammonium salt of meloxicam;
(c) a binder;
(d) a sugar or sweetener; and
(e) a carrier,
   and optionally a flavoring agent and optionally other excipients, processes for preparing them and their use for treating respiratory or inflammatory complaints in mammals.

### RELATED APPLICATIONS

This application claims benefit of U.S. Ser. No. 60/______, filed Oct. 2, 2003, and claims priority to German Application No. 102 50 081.9, filed Oct. 25, 2002, each of which is hereby incorporated by reference in its entirety.

### FIELD OF THE INVENTION

The present invention relates to meloxicam granules which dissolve rapidly in water, containing meloxicam, a salt forming agent which forms the meglumine, sodium, potassium, or ammonium salt of meloxicam, binders, a sugar or sweetener, a carrier, optionally a flavoring, and optionally other excipients, processes for preparing them, and their use for treating respiratory or inflammatory complaints in mammals.

### BACKGROUND OF THE INVENTION

Meloxicam (4-hydroxy-2-methyl-N-(5-methyl-2-thiazolyl)-2H-1,2-benzothiazine-3-carboxamide-1,1-dioxide) is an active substance which belongs to the group of NSAIDs (non-steroidal-antiinflammatory drugs). Meloxicam and the sodium and meglumine (N-methyl-D-glucamine) salt thereof are described in EP-A-0 002 482. EP-A-0 945 134 discloses the pH-dependent solubility characteristics of meloxicam and its salts, i.e., the sodium salt, the ammonium salt, and the meglumine salt, in aqueous solution. According to this, meloxicam is an active substance which does not dissolve readily in water. The meloxicam salts, particularly the meglumine salt, exhibit improved solubility as the pH increases between 4 and 10, as shown in Table 1 of EP-A-0 945 134.

It is known that administering medicaments to sick animals, particularly those suffering from fever, can be done particularly easily and successively through their drinking water. Administering to their food can also make it easier to give the medicament to the animal. It is known from EP-A-0 945 134 that meloxicam and meglumine cannot easily be compressed. The aim of the present invention is therefore to develop a granulated form of meloxicam which can be administered to the animals by mixing it into their drinking water or as a food supplement.

### DESCRIPTION OF THE INVENTION

Surprisingly, meloxicam granules have been discovered which can easily be produced by a fluidized bed method and which, when dissolved in water, form a drinking water solution which is stable over at least 48 hours. It was also found that these granules can be added to the animals' food.

The invention therefore relates to water soluble granules containing meloxicam, a salt forming agent which forms the meglumine, sodium, potassium, or ammonium salt of meloxicam, binders, a sugar or sweetener, a carrier, optionally a flavoring, and optionally other excipients.

The meloxicam granules according to the invention have a number of advantages over existing preparations.

In sick animals, an increased uptake of drinking water can be observed when a drink containing meloxicam is given. Suitable dilution of the dissolved granules allows a variable, precise dosing of the active substance meloxicam. Because of the good solubility of the meloxicam granules according to the invention in water, the effects of meloxicam in the body of the sick animal set in very rapidly. The good flavor of the meloxicam granules also makes it possible to administer them as a food supplement. In addition, the granules according to the invention have very good flow properties, a uniform meloxicam content, they are virtually free from dust and have a narrow particle size distribution of 125 μm to 500 μm. The total solubility of the granules in water ensures optical control of a totally dissolved active substance which is only available for therapeutic use in this form when administered in drinking water. In a preferred embodiment of the invention, the salt forming agent is meglumine. In another preferred embodiment of the invention, the binder may be selected from among hydroxypropylmethylcellulose, polyvinylpyrrolidone, gelatine, starch, and polyethylene glycol ether, preferably hydroxypropylmethylcellulose, polyvinylpyrrolidone, and polyethylene glycol ether, most preferably hydroxypropylmethylcellulose and polyvinylpyrrolidone.

In another preferred embodiment of the invention, the sugar or sweetener may be selected from among sodium saccharine, aspartame, and SUNETT® sweetener (acesulfame potassium, preferably sodium saccharine or aspartame. Particularly preferred according to the invention are meloxicam granules in which the flavoring is selected from among vanilla, honey flavoring, apple flavoring, and contramarum, preferably honey flavoring and apple flavoring. Also particularly preferred are meloxicam granules in which the carrier is selected from among lactose, glucose, mannitol, xylitol, sucrose, and sorbitol, preferably glucose, lactose, or sorbitol, more preferably glucose or lactose, most preferably glucose.

Particularly preferred are meloxicam granules in which the content of meloxicam is between 0.05% and 4%, preferably between 0.1% and 2%, preferably between 0.3% and 1.5%, more preferably between 0.4% and 1%, most preferably 0.6%. Also particularly preferred are meloxicam granules which contain meglumine and meloxicam in a molar ratio of about 9:8 to 12:8, preferably 10:8.

The invention further relates to process for preparing the meloxicam granules according to the invention in which the steps (a) to (c) are carried out successively:
(a) preparing an aqueous granulating liquid containing binder, optionally a sugar or sweetener, meloxicam, meglumine, and/or a flavoring;
(b) spraying the granulating liquid on to a carrier in a topspray fluidized bed method with an air current supplied at a constant temperature from 50° C. to 80° C., preferably 65° C.; and
(c) a subsequent coating process with an aqueous granulating liquid by the topspray fluidized bed method containing a binder, a sugar or sweetener, and/or a flavoring.

In a preferred process according to the invention the granulating liquid is prepared by stirring and heating the components to 70° C. to 100° C., preferably about 90° C.

A particular feature of the meloxicam granules according to he invention is that they have a long term stability of 24 months or more when stored in their original package at room temperature.

A particularly preferred granulated meloxicam preparation contains meloxicam, meglumine, hydroxypropylmethylcellulose, povidone, and glucose monohydrate.

The present invention further relates to the use of meloxicam granules for preparing a pharmaceutical composition for treating pain, inflammation, fever, acute mastitis, diarrhea, lameness, problems of mobility, and respiratory complaints in animals, preferably acute mastitis, diarrhea, lameness, mobility problems, and respiratory complaints, preferably acute mastitis, diarrhea, lameness, mobility problems, and respiratory complaints, most preferably mobility problems or respiratory complaints. The treatment may be given in conjunction with antibiotic treatment.

The formulation according to the invention is suitable for treating animals, preferably mammals, particularly domestic pets or farm animals, such as pigs, horses, cattle, dogs, or cats, preferably pigs or horses.

The meloxicam granules according to the invention are preferably used in amounts corresponding to a dosage range from 0.2 to 1.0 mg of active substance per kg of bodyweight, preferably 0.4 to 0.8 mg/kg of bodyweight, preferably 0.5 to 0.7 mg/kg of bodyweight, more preferably 0.6 mg/kg of bodyweight.

It is also preferable to use the meloxicam granules according to the invention to prepare a pharmaceutical composition which can be administered both in drink and also as a feed supplement.

The formulation according to the invention may contain, as the meloxicam salt, the meglumine, sodium, potassium or ammonium salt, preferably the meloxicam meglumine salt.

The proportion of meglumine is between 0.035% and 2.8%, preferably 0.07% to 1.4%, preferably 0.21% to 1.05%, more preferably 0.28% to 0.7%, particularly about 0.42% in the meloxicam granules. The possible concentrations of sodium, potassium, and ammonium may be calculated accordingly.

The concentration of the binder may be in the range from 20 mg/g to 80 mg/g, preferably 30 mg/g to 70 mg/g, preferably 40 mg/g to 60 mg/g, most preferably 50 mg/g of granules.

The concentration of the sugar may be in the range from 50 mg/g to 150 mg/g, preferably 75 mg/g to 125 mg/g, more preferably about 100 mg/g of granules.

The concentration of the sweetener may be in the range from 1 mg/g to 10 mg/g, preferably 2 mg/g to 5 mg/g, more preferably about 3 mg/g of granules.

The concentration of the carrier may be in the range from 800 mg/g to 985 mg/g, preferably 900 mg/g to 960 mg/g, more preferably about 930 mg/g of granules.

The concentration of the flavoring may be in the range from 0.1 mg/g to 10 mg/g, preferably 0.2 mg/g to 1.0 mg/g, more preferably about 0.5 mg/g of granules.

The packaging material used for the formulation according to the invention may be any of a number of standard commercial materials for granules. These include, for example, plastic containers, e.g., made of HPPE (high pressure polyethylene), aluminum bags, or paper bags with an aluminum lining.

The meloxicam granules are produced by the top spray fluidized bed method. In this, first of all an aqueous granulating liquid solution consisting of about 50 to 70 g/kg of binder, such as PVP 25000, hydroxypropylmethylcellulose or Macrogol 6000, preferably hydroxypropylmethylcellulose, and/or about 1 to 5 g/kg of sweeteners such as SUNETT® sweetener (acesulfame potassium) or Na saccharine, preferably SUNETT® sweetener (acesulfame potassium), and/or about 0.5 to 2.5 g of flavoring, such as vanilla, honey, flavoring 203180, or contramarum, preferably honey, about 10 g to 15 g of meloxicam (peg milled) and about 7 g to 11 g of meglumine is produced with stirring by heating to about 70° C. to 100° C.

The granulating liquid is then sprayed on to a carrier such as lactose, glucose, or sorbitol, preferably glucose, by a counter flow process (top spray process). This is done, for example, using a two-component nozzle, spraying at a constant air pressure at about 50° C. to 80° C., preferably at about 65° C. The coating process may then be carried out using a second aqueous granulating liquid. In order to prepare a solution ready for use, a stock solution should be dissolved completely in water. Then the stock solution may be adjusted to the desired concentration for use by mixing with water. To increase safety in use, the granules may have water soluble color markings.

The meloxicam granules according to the invention will be illustrated by the examples that follow. The skilled person will be aware that these examples are intended solely as an illustration and should not be regarded as limiting the invention.

### EXAMPLE 1

### 0.6% Meloxicam Granules

| Recipe: | g/100 g |
|---|---|
| Meloxicam | 0.6 |
| Meglumine | 0.42 |
| Hydroxypropylmethylcellulose | 3.00 |
| Povidone | 2.00 |
| Glucose monohydrate | 93.98 |

### EXAMPLE 2

### 1.2% Meloxicam Granules

| | |
|---|---|
| Meloxicam | 1.2 |
| Meglumine | 0.84 |
| Hydroxypropylmethylcellulose | 3.00 |
| Collidone 25 | 2.0 |
| Glucose Monohydrate | 92.96 |

### EXAMPLE 3

### 0.6% Meloxicam Granules

| | |
|---|---|
| Meloxicam | 0.6 |
| Meglumine | 0.42 |
| Pharmacoat 606 | 4.0 |
| Macrogol 6000 | 1.0 |
| Acesulfame K | 0.3 |
| Lactose | 93.68 |

### EXAMPLE 4

### 0.6% Meloxicam Granules

| | |
|---|---|
| Meloxicam | 0.6 |
| Meglumine | 0.42 |
| Pharmacoat 606 | 4.75 |
| Macrogol 6000 | 0.25 |
| Acesulfame K | 0.3 |
| Liquid vanilla flavoring | 0.05 |
| Lactose | 93.63 |

Bright yellow free flowing meloxicam granules corresponding to Examples 1 to 4 may be prepared as follows:

The granules are stored for 3 months at 25° C. at a relative humidity of 60%. No significant changes were observed in terms of the active substance content, the water content (according to Karl-Fischer), the visual solubility characteristics, the pH in demineralized water, and the visual wetability. In order to determine the visual solubility characteristics, 5 g of the granules were dissolved in 100 mL of demineralized water at ambient temperature. After about 1 minute, a clear yellowish solution was obtained.

We claim:

1. Water soluble meloxicam granules comprising:
(a) meloxicam;
(b) a salt forming agent which forms the meglumine, sodium, potassium, or ammonium salt of meloxicam;
(c) a binder;
(d) a sugar or sweetener; and
(e) a carrier,
and optionally a flavoring agent and optionally other excipients.

2. The water soluble meloxicam granules according to claim 1, wherein the salt forming agent is meglumine.

3. The water soluble meloxicam granules according to claim 1, wherein the binder is selected from hydroxypropylmethylcellulose, polyvinylpyrrolidone, gelatine, starch, or polyethylene glycol ether.

4. The water soluble meloxicam granules according to claim 2, wherein the binder is selected from hydroxypropylmethylcellulose, polyvinylpyrrolidone, gelatine, starch, or polyethylene glycol ether.

5. The water soluble meloxicam granules according to claim 1, wherein the sugar or sweetener is selected from sodium saccharine, aspartame, and acesulfame potassium.

6. The water soluble meloxicam granules according to claim 2, wherein the sugar or sweetener is selected from sodium saccharine, aspartame, and acesulfame potassium.

7. The water soluble meloxicam granules according to claim 3, wherein the sugar or sweetener is selected from sodium saccharine, aspartame, and acesulfame potassium.

8. The water soluble meloxicam granules according to claim 4, wherein the sugar or sweetener is selected from sodium saccharine, aspartame, and acesulfame potassium.

9. The water soluble meloxicam granules according to claim 1, wherein the flavoring agent is selected from vanilla, honey flavoring, apple flavoring, or contramarum.

10. The water soluble meloxicam granules according to claim 2, wherein the flavoring agent is selected from vanilla, honey flavoring, apple flavoring, or contramarum.

11. The water soluble meloxicam granules according to claim 3, wherein the flavoring agent is selected from vanilla, honey flavoring, apple flavoring, or contramarum.

12. The water soluble meloxicam granules according to claim 4, wherein the flavoring agent is selected from vanilla, honey flavoring, apple flavoring, or contramarum.

13. The water soluble meloxicam granules according to claim 1, wherein the carrier is selected from lactose, glucose, mannitol, xylitol, sucrose, and sorbitol.

14. The water soluble meloxicam granules according to claim 2, wherein the carrier is selected from lactose, glucose, mannitol, xylitol, sucrose, and sorbitol.

15. The water soluble meloxicam granules according to claim 3, wherein the carrier is selected from lactose, glucose, mannitol, xylitol, sucrose, and sorbitol.

16. The water soluble meloxicam granules according to claim 4, wherein the carrier is selected from lactose, glucose, mannitol, xylitol, sucrose, and sorbitol.

17. The water soluble meloxicam granules according to claim 1, wherein the proportion of meloxicam is between 0.05% and 4%.

18. The water soluble meloxicam granules according to claim 2, wherein the molar ratio of meglumine to meloxicam is 9:8 to 12:8.

19. The water soluble meloxicam granules according to claim 2, wherein the molar ratio of meglumine to meloxicam is about 10:8.

20. Water soluble meloxicam granules comprising meloxicam, meglumine, hydroxypropylmethylcellulose, povidone, and glucose monohydrate.

21. A process for preparing the water soluble meloxicam granules according to claim 1, the process comprising:
(a) preparing an aqueous granulating liquid containing the binder, the sugar or sweetener, the meloxicam, the salt forming agent, and/or the flavoring agent;
(b) spraying the granulating liquid on to a carrier in a topspray fluidized bed method with a supply of air at a constant temperature of 50° C. to 80° C.; and
(c) performing a coating process with an aqueous granulating liquid by the topspray fluidized bed method containing the binder, the sugar or sweetener, and/or the flavoring agent.

22. A process for preparing the water soluble meloxicam granules according to claim 2, the process comprising:
(a) preparing an aqueous granulating liquid containing the binder, the sugar or sweetener, the meloxicam, the meglumine, and/or the flavoring agent;
(b) spraying the granulating liquid on to a carrier in a topspray fluidized bed method with a supply of air at a constant temperature of 50° C. to 80° C.; and
(c) performing a coating process with an aqueous granulating liquid by the topspray fluidized bed method containing the binder, the sugar or sweetener, and/or the flavoring agent.

23. The process according to claim 21, wherein the granulating liquid is prepared by stirring and heating the components to 70° C. to 100° C.

24. The process according to claim 22, wherein the granulating liquid is prepared by stirring and heating the components to 70° C. to 100° C.
